(19) United States
(12) Patent Application Publication
(10) Pub. No.: US 2018/0021023 A1
(43) Pub. Date: Jan. 25, 2018

(54) Ultrasound Imaging Device and Ultrasound Signal Processing Method

(71) Applicant: Hitachi, Ltd., Chiyoda-ku, Tokyo (JP)

(72) Inventors: Teiichiro IKEDA, Tokyo (JP); Hiroshi KURIBARA, Tokyo (JP); Chizue ISHIHARA, Tokyo (JP); Mayumi SUZUKI, Tokyo (JP); Misaki HIROSHIMA, Tokyo (JP)

(73) Assignee: Hitachi, Ltd., Chiyoda-ku, Tokyo (JP)

(21) Appl. No.: 15/547,903

(22) PCT Filed: Jan. 5, 2016
(86) PCT No.: PCT/JP2016/050114

§ 371 Date: Aug. 1, 2017

(30) Foreign Application Priority Data
Feb. 4, 2015 (JP) 2015-020745

(51) Int. Cl. A61B 8/08; G01S 15/89

(52) U.S. Cl. CPC A61B 8/5207; G01S 15/8997; G01S 7/52095

(57) ABSTRACT

There is realized an ultrasonic diagnostic apparatus that eliminates the restriction on the image generation in ultrasonic diagnostic apparatuses imposed by generation of no more than one delay curve, and enables imaging with higher resolution and higher speed.

A received signal processor 12 comprises delayers 13, 14-1, and 14-2 disposed for every reception channel, and a synthesiser 60. The first delayer 13 delays received signals produced from a transmission beam 31 by a first delay time for phasing the received signals for a predetermined reception focus. The second delayer 14-1 delays received signals produced from a sound wave of a predetermined phase different from the phase of the transmission beam 31 by a second delay time for phasing the received signals for the same reception focus. The synthesiser 60 adds first phased signals generated by the first delayer, and second phased signals generated by the second delayer 14-1.

### TECHNICAL FIELD

The present invention relates to an ultrasonic imaging technique for obtaining an image of the inside of a subject using an ultrasonic wave.

### BACKGROUND ART

Ultrasonic imaging technique is a technique for non-invasively imaging the inside of a subject such as human body using an ultrasonic wave (inaudible sound wave, generally a sound wave of such a high frequency as 20 kHz or higher).

There are two kinds of methods of transmitting ultrasonic beams from an ultrasound probe to a subject, i.e., diffusion transmission in which ultrasonic beams spreading in a fan shape are transmitted, and convergence transmission in which ultrasonic beams are transmitted so as to converge at a transmission focus of ultrasonic beams set in the inside of a subject.

Since transmission and reception of ultrasonic waves by an ultrasonic imaging apparatus are performed with an array having a limited opening diameter, they are influenced by diffraction of ultrasonic waves with an edge of the opening, and it is difficult to improve resolution for the azimuth direction. This problem can be solved if an infinitely long array can be prepared, but it is impossible to actually realize it. Therefore, in recent years, channel domain phasing technique is actively studied in order to improve resolution for the azimuth direction, and novel phasing schemes such as adaptive beamformer and aperture synthesis have been actively reported.

The aperture synthesis will be briefly explained. First, by imparting delay times to signals received by a plurality of devices constituting the ultrasound probe, they are virtually focused at a certain point, and then added to obtain phased signals. The aperture synthesis is performed by synthesizing these phased signals and phased signals obtained by other one or more times of transmission and reception for the same point to superimpose them.

By the aperture synthesis, phased signals obtained for a certain point by transmissions and receptions performed in different directions with an ultrasound probe can be superimposed, and therefore it is expected to, for example, provide higher resolution of point image, and impart robustness against heterogeneity. Furthermore, since processing gain is improved by the superimposition processing, the number of transmission of ultrasonic waves can be reduced compared with that of usual cases, and therefore it can also be applied to high-speed imaging.

Patent document 1 discloses a technique that, in ultrasonic imaging using an ultrasonic diagnostic apparatus that performs convergence transmission, the aperture synthesis is performed by using an improved virtual sound source method. Specifically, in a region in which energy of an ultrasonic beam converges at a focus (region A indicated in FIG. 2 of Patent document 1), the aperture synthesis is performed by regarding the focus as the virtual sound source, and in a region around the foregoing region in which ultrasonic energy diffuses (regions B and C), the aperture synthesis is performed by regarding that a spherical wave has been transmitted from the end of the probe.

### PRIOR ART REFERENCE

### Patent Document

Patent document 1: Japanese Patent Unexamined Publication (KOKAI) No. 10-277042

### SUMMARY OF THE INVENTION

### Object to be Achieved by the Invention

As described in Patent document 1, by obtaining delay times by the virtual sound source method in a transmission beam irradiation region (region in which energy of ultrasonic beam converges), and obtaining the delay times by regarding that a spherical wave is transmitted from an end of the probe in a region other than the transmission beam irradiation region (region in which ultrasonic beam energy diffuses), phased signals can be obtained for a point out of the transmission beam irradiation region. Therefore, reception scanning lines can be set also for the outside of the transmission beam irradiation region.

However, when delay time for a point on a reception scanning line outside the transmission beam irradiation region is obtained on the basis of waveform of a spherical wave that is regarded to be transmitted from the end of the probe according to the technique of Patent document 1, advancing directions of the spherical waves transmitted from the both ends of the probe cross with each other around the depth of the transmission focus, and therefore the waveform of the spherical wave used for the calculation of the delay time must be changed from that of one of the spherical waves transmitted from the left and right ends of the probe to that of the other spherical wave. Thus, there arises a problem that the curve representing change of the delay time for the depth direction on the reception scanning line is interrupted around the depth of the transmission focus.

In ultrasonic diagnostic apparatuses, the delay addition processing is generally performed by using no more than one delay curve for every reception scanning line, which is also applied to the technique of Patent document 1. In the convergence transmission, the delay curve becomes discontinuous around the depth of the focus.

As a result, pixel value of the generated sonogram becomes discontinuous around the transmission focus, which generates artifacts around the depth of the transmission focus.

An object of the present invention is to eliminate the restriction on the image generation with ultrasonic diagnostic apparatuses imposed by generating no more than one delay curve, and thereby realize an ultrasonic diagnostic apparatus that enables imaging with higher resolution and higher speed.

### Means for Achieving the Object

The ultrasonic imaging apparatus of the present invention comprises a received signal processor that processes received signals obtained by receiving sound waves transmitted from a subject, to which a transmission beam imparted with such a predetermined phase delay that it focuses on a predetermined transmission focus has been transmitted, with a plurality of reception channels to obtain phased signals. The received signal processor comprises delayers, which are provided in a number of 2 or larger for each reception channel, and a synthesiser.

Among the plurality of the delayers, a first delayer delays the received signals by a first delay time for phasing received signals produced from the transmission beam for a predetermined reception focus. A second delayer delays the received signal by a second delay time for phasing received signals produced from a sound wave of a predetermined phase different from the phase of the transmission beam for the same reception focus. The synthesiser adds first phased signals generated by the first delayer through the aforementioned delaying, and second phased signals generated by the second delayer through the aforementioned delaying.

### Effect of the Invention

According to the present invention, phased signals can be obtained not only for received signals originating in a transmission beam, but also for received signals originating in a sound wave of a phase different from that of the transmission beam, and an image can be generated by using the both signals. Therefore, image quality of higher resolution can be obtained. Further, phased signals can also be obtained for a part outside the transmission beam, and therefore high speed imaging can be realized.

### BRIEF DESCRIPTION OF THE DRAWINGS

FIG. 1 An explanatory drawing for explaining a transmission beam (direct beam) 31 and non-direct beams 33-1 and 33-2.

FIG. 2A: An explanatory drawing showing a transmission beam 31 and a sound axis 36a, FIG. 2B: a graph showing waveforms of waves that arrive at various positions for the depth direction of the sound axis 36a at various times.

FIG. 3 A block diagram showing the configuration of the ultrasonic imaging apparatus of the first embodiment.

FIG. 4 An explanatory drawing showing the shape of the transmission beam 31 and a plurality of reception scanning lines.

FIG. 5 A block diagram showing the configuration of the received signal processor of the ultrasonic imaging apparatus of the second embodiment.

FIG. 6A: An explanatory drawing showing examples of masks for direct beam and non-direct beam according to the second embodiment, FIG. 6B: an explanatory drawing showing weight distributions for direct beam and non-direct beam.

FIG. 7A: a block diagram showing a configuration that an envelope demodulator and an LOG compressor are provided as subsequent stages of the RF signal processor 15 according to the second embodiment, FIG. 7B: a block diagram showing a configuration that the envelope demodulator is provided as a preceding stage of the RF signal processor 15, and the LOG compressor is provided as a subsequent stage of the same, FIG. 7C: a block diagram showing a configuration that the envelope demodulator and the LOG compressor are provided as preceding stages of the RF signal processor 15.

FIGS. 8A and 8B: Explanatory drawings showing a region for which phased signals based on a direct beam and phased signals based on a non-direct beam are superimposed according to the second embodiment.

FIG. 9 A flowchart showing operations of the ultrasonic diagnostic apparatus of the first embodiment.

FIG. 10 An explanatory drawing showing operations of the ultrasonic diagnostic apparatus of the second embodiment.

FIG. 11 A block diagram showing the apparatus configuration in the case of realizing the operations of the ultrasonic diagnostic apparatus of the second embodiment with hardware.

FIG. 12 A block diagram showing the apparatus configuration in the case of realizing the operations of the ultrasonic diagnostic apparatus of the second embodiment with software.

FIG. 13A: An explanatory drawing showing the transmission beam 31 and reception scanning lines 36, FIG. 13B: a graph showing times (propagation distances) until a direct beam and a non-direct beam reach various positions on a reception scanning line coinciding to the sound axis, FIGS. 13C and 13D: graphs showing times until a direct beam and a non-direct beam reach various positions on a reception scanning line remote from the sound axis, and the delay time obtained according to the virtual sound source method.

FIG. 14 A block diagram showing the position of the synthesiser 60 according to the third embodiment.

FIG. 15A: A block diagram for the case where a synthesis area setter 58 is provided in the third embodiment, FIG. 15B: a block diagram for the case where a weighting part 59 is provided in the third embodiment.

FIG. 16 A block diagram showing the configuration of the comparative example in which phased signals (LRI) are obtained for a plurality of reception scanning lines by parallel beam processing.

### MODES FOR CARRYING OUT THE INVENTION

The ultrasonic imaging apparatus according of the present invention will be explained.

### (Principle of the Present Invention)

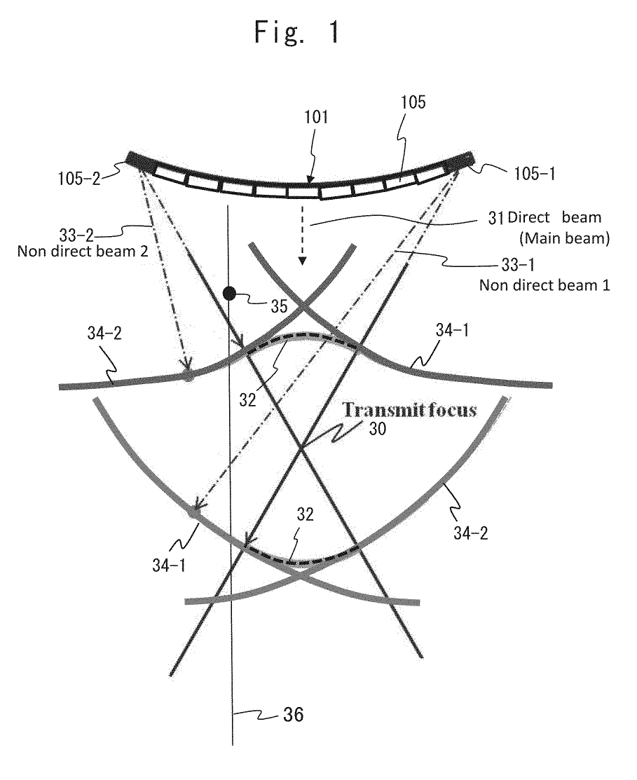

First, the principle of the present invention will be explained. As shown in FIG. 1, the ultrasonic imaging apparatus transmits ultrasonic waves each delayed by a predetermined delay amount so that they focus on a predetermined transmission focus 30 from a plurality of transmission channels 105 of an ultrasonic device array 101. As a result, the ultrasonic waves transmitted from the plurality of the transmission channels 101 interfere with one another to form an interference wave (transmission beam 31). The wave front of the transmission beam 31 is a wave front 32. In a subject, diffracted waves (spherical waves) 33-1 and 33-2 of a phase different from that of the transmission beam 31 also propagate. In the following explanation, sound waves transmitted from transmission channels 105-1 and 105-2 at the both ends of the ultrasonic device array, respectively, are used as the diffracted wave 33-1 and 33-2 as examples.

An ultrasonic wave (transmission beam) 31 of which phase is delayed for the convergence at a transmission focus 30 is also referred to as “direct beam (wave)”. Diffracted waves (spherical waves) 33-1 and 33-2 of a phase different from that of the transmission beam (direct beam) 31 are also referred to as “non-direct beam (wave)”. Both or either one of the non-direct beams 33-1 and 33-2 is referred to as non-direct beam 33.

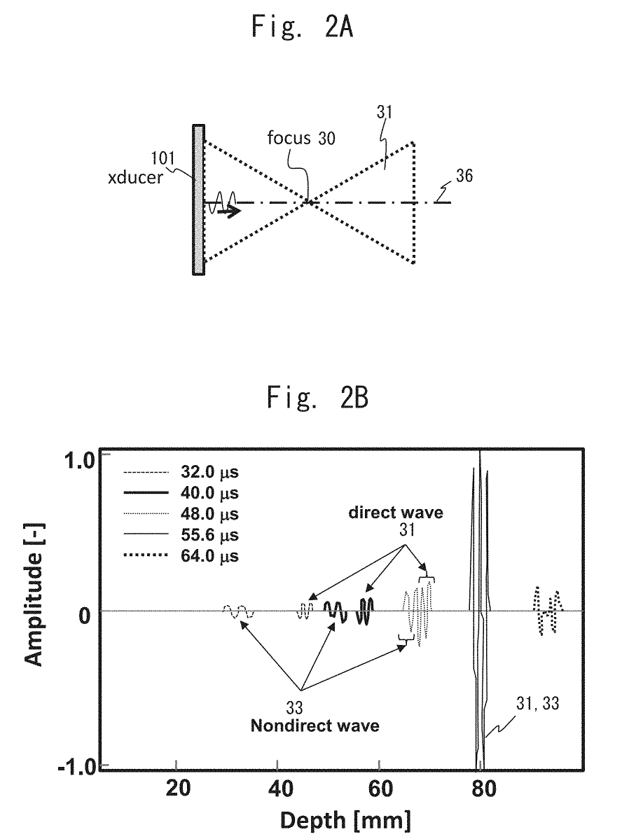

FIG. 2A shows the transmission beam (direct beam) 31 transmitted from the ultrasonic device array 101, and the central axis (sound axis) 36a thereof. FIG. 2B shows the results of simulation for obtaining waveforms of the direct beam 31 and non-direct beam 33 that reached various depths along the sound axis 36a of transmission beam 31 after predetermined times (32, 40, 48, 55.6, and 64 microseconds) after the transmission. The depth of the transmission focus 30 is 80 mm. From FIG. 2B, it can be seen that two waveforms are observed as a set for the same time at different depths. In each set of waveforms of the same time, the wave near the transmission focus 30 is the direct beam 31, and the wave distant from the transmission focus 30 is the non-direct beam 33. At the transmission focus 30 (time of 55.6 μs), the direct beam 31 and the non-direct beam 33 reached at the same time, and form one sound pressure waveform. The non-direct beam 33 shown in FIG. 2B has a waveform formed by superimposition of the non-direct beams 33-1 and 33-2 shown in FIG. 1 on the sound axis 36a.

From FIG. 2B, it can be confirmed that, on the sound axis 36a, two waves (direct beam 31 and non-direct beam 33) have actually propagated to each depth excluding the transmission focus 30, and they show sound pressures of similar order. Since FIG. 2B shows waveforms on the sound axis 36a, the non-direct beams 33-1 and 33-2 superimpose with each other to form the non-direct beam 33. However, at a position distant from the sound axis 36a, three kinds of waves, the direct beam 31, the non-direct beam 33-1, and the non-direct beam 33-2, propagate.

In conventional ultrasonic imaging apparatuses, beamforming is performed by using such a delay time for performing phasing for only the direct beam 31, therefore an image is generated by using only the information obtained with the direct beam 31, and the information obtained with the non-direct beams 33-1 and 33-2 is not used for the image generation. According to the present invention, by performing the image generation using information based on not only the direct beam 31, but also at least one of the non-direct beams 33-1 and 33-2, the resolution of the image is improved, and high-speed image generation is enabled.

### First Embodiment

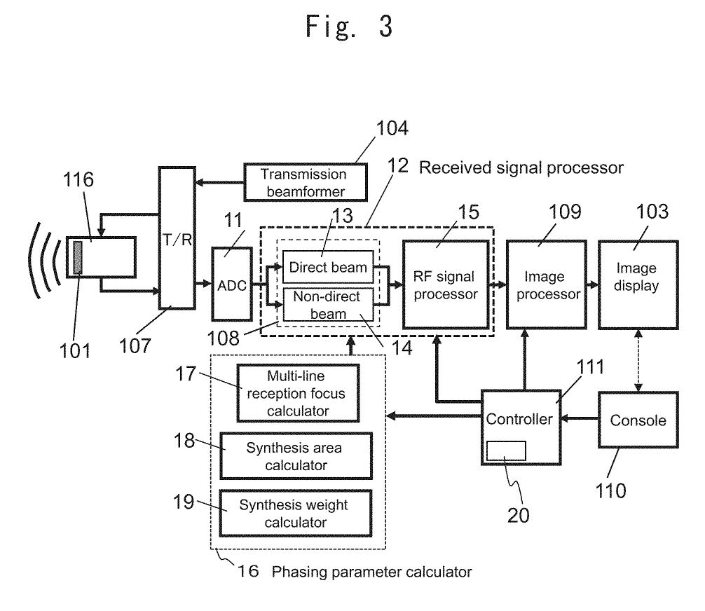

As shown in FIG. 3, the ultrasonic diagnostic apparatus of the first embodiment uses the received signal processor 12 comprising two or more delayers 13 and 14 for each reception channel. Among two or more of the delayers 13 and 14, the first delayer 13 carries out phasing of the received signals produced from the transmission beam (direct beam) 31 for a predetermined reception focus 35 by delaying the received signals by a predetermined first delay time. The first delay time is set so that received signals of reflected waves of the direct beam 31 reflected by the subject are phased. The second delayer 14 carries out phasing of the received signals produced from the sound wave (non-direct beam) 33 of a predetermined phase different from the phase of the transmission beam 31 for the reception focus 35 by delaying the received signals by a second delay time. The second delay time is set so that the received signals of reflected waves of one of the non-direct beams 33-1 and 33-2 are phased.

Further, the ultrasonic imaging apparatus adds the first phased signals generated by the first delayer 13 by the delaying, and the second phased signal generated by the second delayer 14 by the delaying in a synthesiser.

By generating an image using the added phased signals, an image can be generated by using information obtained with not only the direct beam 31, but also the non-direct beam 33.

As described above, the ultrasonic imaging apparatus of this embodiment uses the processing of phasing the received signals for the reception focus 35 in order to extract the information based on the direct beam 31 and the information based on the non-direct beam 33 from the received signals. Since wave fronts 34-1 and 34-2 of the non-direct beams 33-1 and 33-2 differ from that of the direct beam 31 (wave front 32) as shown in FIG. 1, the phases thereof also differ. Therefore, phases of the received signals based on the direct beam 31 and the received signals based on the non-direct beams 33-1 and 33-2 included in the received signals received by the reception channels of the ultrasonic device array 101 differ from each other. That is, for a certain reception focus 35, the first delay time for carrying out phasing of the received signals of the reflected wave of the direct beam 31 has a value different from that of the second delay time for carrying out phasing of the received signals of the reflected waves of the non-direct beams 33-1 and 33-2. Therefore, by providing two or more delayers 14 and 15 for each reception channel, and performing delay processing in the first delayer 14 using the first delay time for carrying out phasing of the received signals of the reflected waves of the direct beam 31, received signals based on the direct wave 31 can be extracted.

Further, by performing the delay processing in the second delayer 15 using the second delay time for carrying out phasing of the received signals of the reflected waves of one of the non-direct beams 33-1 and 33-2, received signals based on the non-direct beam 33-1 or 33-2 can be extracted.

According to this embodiment, both the information on the reception focus obtained with the direct beam 31 and the information on the reception focus obtained with the non-direct beam 33 can be used, and therefore high resolution and high speed imaging is enabled. Further, artifacts resulting from discontinuity of the change of the delay time along the depth direction around the transmission focus occurring when only the direct beam 31 is used can be suppressed by using both the direct wave 31 and non-direct wave 33.

By additionally using the non-direct beam 31, a region that cannot be imaged with only the direct wave 31 can be imaged, therefore the imaging region obtainable by one time of sound wave transmission is widened, and image formation amount per unit time can be improved. That is, high speed ultrasonic imaging is enabled. Further, since an imaging point (reception focus) conventionally imaged only with the direct beam 31 can be simultaneously imaged by using the non-direct beam 33, multi-look imaging, in which a certain imaging point is imaged by using sound waves reached from a plurality of directions, can be enabled even with not more than one time of transmission. Therefore, an image of high resolution can be obtained. By performing the aperture synthesis, which is a kind of multi-look imaging, as a subsequent stage using the method of this embodiment, two-stage multi-look imaging is enabled, and thus it becomes possible to obtain an image of further higher resolution.

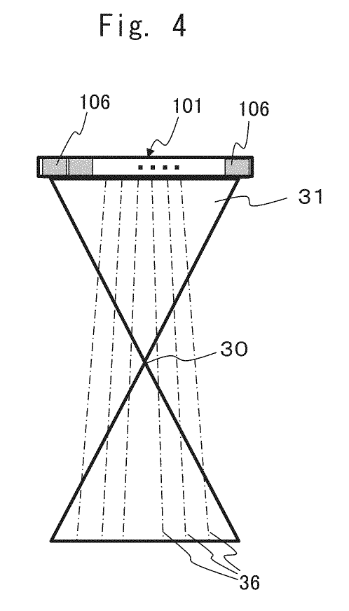

The ultrasonic imaging apparatus of this embodiment can generate phased signals for any region which either one of the direct beam 31 and the non-direct beams 33-1 and 33-2 reaches. Therefore, as shown in FIG. 4, a reception scanning line 36 can be set not only inside the region to which the direct beam (transmission beam) 31 is transmitted, but also outside the region, and phased signals can be obtained for a reception focus on the reception scanning line 36. As a result, the received signal processor 12 can set a plurality of reception scanning lines for one time of transmission of the transmission beam 31, and generate phased signals for a plurality of reception focuses on the plurality of the reception scanning lines, and therefore it can generate an image at high speed.

Although FIGS. 1 and 2 also depict configurations other than those explained above, the ultrasonic imaging apparatus of the first embodiment does not need to comprise these configurations. The configurations except for those explained above will be explained as configurations of the ultrasonic imaging apparatus of the second embodiment.

### Second Embodiment

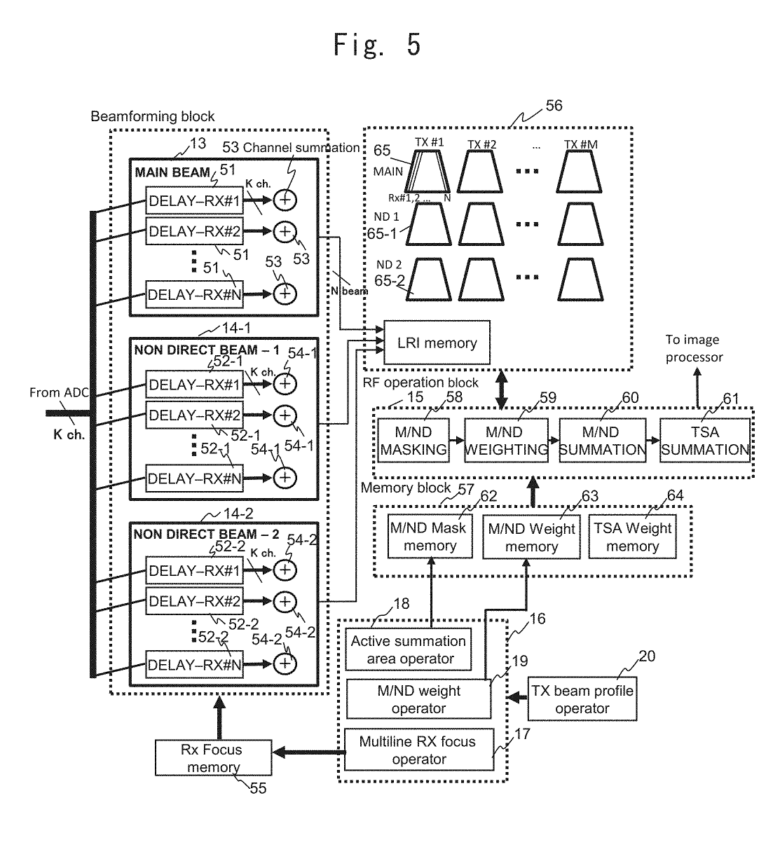

The ultrasonic imaging apparatus of the second embodiment is explained below. As shown in FIG. 5, the ultrasonic imaging apparatus of the second embodiment comprises three delayers 13, 14-1, and 14-2, and performs phasing processing for each of the direct beam 31, and non-direct beams 33-1 and 33-2. The same configurations of the second embodiment as those of the first embodiment are indicated with the same numerical notations.

As shown in FIG. 3, the ultrasonic imaging apparatus of the second embodiment comprises an ultrasound probe 116, a transmission beamformer 104, a received signal processor 12, a phasing parameter calculator 16, an image processor 109, a controller 111, a console 110, and an image display 103. The ultrasound probe 116 comprises an ultrasonic device array 101 on which ultrasonic devices are arranged.

Between the ultrasound probe 116, and the transmission beamformer 104 and the received signal processor 12, a transmission/reception separation circuit (T/R) 107 is disposed. Between the transmission/reception separation circuit 107 and the received signal processor 12, an analogue/digital converter 11 is disposed.

The console 110 receives input of the position of the transmission focus 30, transmission frequency, number of times of transmission, imaging area, etc. from an operator. There may also be employed a configuration that the console receives settings linked with a specific purpose of the operator such as setting of imaging mode, setting of imager, and setting of application, and according to the input of setting of imaging mode, setting of imager, and setting of application from the console 110, specific transmission frequency, number of times of transmission, and imaging area are determined by the controller 111 so as to correspond to the inputted settings of them. That is, there may be employed a configuration that the transmission frequency, number of times of transmission, and imaging area are set implicitly for the operator.

The transmission beamformer 104 generates transmission signals of phases delayed for every transmission channel so that the ultrasonic waves should converge at the position of the transmission focus 30 received from the controller 111, and sends them to each transmission channel 105 of the ultrasonic device array 101. As a result, ultrasonic waves are transmitted from each of the plurality of the transmission channels 105 of the ultrasonic device array 101, they interfere one another to form the transmission beam (direct beam) 31, and this transmission beam 31 propagates the imaging area of the subject. At the same time, a part of the ultrasonic waves transmitted from the transmission channels 105-1 and 105-2 at the both ends of the ultrasonic device array 101 that does not contribute to the formation of the transmission beam 31 propagate the imaging area of the subject as diffracted waves (non-direct beams) 33-1 and 33-2.

The received signal processor 12 comprises a reception beamformer 108, an RF signal processor 15, a reception focus memory 55, an LRI (low resolution image) memory 56, and a memory 57 for synthesis as shown in FIGS. 3 and 5.

According to the second embodiment, the reception beamformer 108 comprises a first delayer 13 and two second delayers 14-1 and 14-2, as shown in FIG. 5. These three delayers 13, 14-1, and 14-2 comprise delay circuit sets 51, 52-1, and 52-2, and adders 53, 54-1, and 54-2, respectively. The delay circuit sets 51, 52-1, and 52-2 each comprise delay circuits in a number (K) equal to the number (K) of the reception channels 106 of the ultrasonic device array 101. The delay circuits in the number of K delay the received signals outputted by K of the reception channels 106 by the delay time stored in the reception focus memory 55 for each reception focus. The adders 53, 54-1, and 54-2 add the outputs of K of the delay circuits of the delay circuit sets 51, 52-1, and 52-2, respectively.

It is sufficient that one each of the delay circuit set 51, 52-1, and 52-2 and one each of the adders 53, 54-1, and 54-2 are provided in the delayer 13, 14-1, and 14-2.

However, in the example mentioned for explaining this embodiment, in order to perform the phasing processing in parallel for a plurality (N) of reception scanning lines, N of the delay circuit sets 51, 52-1, and 52-2, and N of the adders 53, 54-1, and 54-2 are provided in the delayers 13, 14-1, and 14-2, respectively.

The plurality (N) of the reception scanning lines may be generated by using time sharing. Namely, the delay circuit sets 51, 52-1, and 52-2 in a number of L smaller than N, and L of the adders 53, 54-1, and 54-2 are provided in the delayers 13, 14-1, and 14-2, respectively, and delay circuit and adder where the operation is once performed for a certain reception scanning line can be repeatedly used so long as there is blank time until next reception data are received, and may perform the delay operation and addition operation again for a different reception scanning line.

Although it is also possible to employ a configuration that the reception focus memory 55 preliminarily stores delay times separately obtained beforehand, in the example mentioned for the explanation of this embodiment, the delay time obtained by a multi-line reception focus operator 17 to be explained later by calculation is stored for every reception focus of the reception scanning line.

The LRI memory 56 successively stores phased signals successively outputted by N of the adders 53, 54-1, and 54-2 in each of the delayer 13, 14-1, and 14-2 for each reception focus so that they are associated with N of the scanning lines. As a result, phased signals for a predetermined number of reception focuses on N of the reception scanning lines are stored, and one LRI (low resolution image) is stored by one time of transmission in each of the delayers 13, 14-1, and 14-2. That is, a low resolution image 65 based on the direct beam 31 is stored from the phased signals outputted by the delayer 13, and low resolution images 66-1 and 66-2 based on the non-direct beams 33-1 and 33-2 are stored from the phased signals outputted by the delayers 14-1 and 14-2, respectively.

When one each of low resolution images 65, 66-1, and 66-2 are stored in the LRI memory 56 in each of the delayers 13, 14-1, and 14-2 by one time of transmission, the RF signal processor 15 can add the phased signals based on the direct beam 31 and the phased signals based on the direct beams 33-1 and 33-2. In this embodiment, in order to further perform inter-transmission aperture synthesis processing, there is employed a configuration that the low resolution images 65, 66-1, and 66-2 for M times of transmission required for the inter-transmission aperture synthesis are stored as shown in FIG. 5.

The RF signal processor 15 comprises a synthesis area setter 58, a weighting part 59, a synthesiser 60, and an inter-transmission aperture synthesiser 61. The memory 57 for synthesis comprises a mask memory 62 that stores masks for determining an area for which phased signals (low resolution image) are synthesized, a weight memory 63 that stores weights to be used at the time of adding phased signals (low resolution image) of a corresponding reception focus, and an inter-transmission aperture synthesis memory 64 that stores inter-transmission weights used for the inter-transmission aperture synthesis.

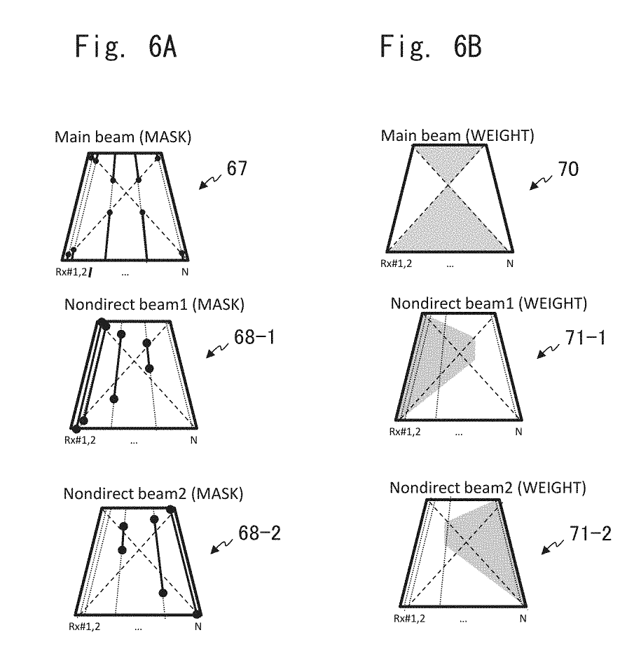

As shown in FIG. 6A, for example, the mask memory 62 stores a mask 67 for direct beam and masks 68-1 and 68-2 for non-direct beam. The weight memory 63 stores different weights varying depending on, for example, position of low resolution image, for example, weight distribution 70 for direct beam and weight distributions 71-1 and 71-2 for non-direct beam, as shown in FIG. 6B.

The synthesis area setter 58 sets the masks 67, 68-1, and 68-2 read from the mask memory 62 for the respectively corresponding low resolution images 65, 66-1, and 66-2.

The weighting part 59 performs weighting of the phased signals of the low resolution images 65, 66-1, and 66-2 in the regions in which the masks 67, 68-1, and 68-2 are set by the synthesis area setter 58 with the weight distributions 70, 71-1, and 71-2 at the positions corresponding in terms of weight stored in the weight memory 59. The synthesiser 60 adds the phased signals of each of the low resolution images 65, 66-1, and 66-2 weighted by the weighting part 59 for corresponding reception focuses. An image is thereby formed, in which the phased signals based on the direct beam 31 and the phased signals based on the non-direct beams 33-1 and 33-2 are synthesized. The synthesized image is stored in the memory build in the synthesiser 60. The synthesis area setter 58, weighting part 59, and synthesiser 60 perform these processings for each of the low resolution images for M times of transmission to obtain M of synthesized images for M times of transmission.

After the setting of the masks by the synthesis area setter 58, data obtained after the mask setting may be temporarily stored in the LRI memory 56. Similarly, the low resolution images weighted by the weighting part 59 may be temporarily stored in the LRI memory. M of low resolution images for M times of transmission may be synthesized in the synthesiser 60 by reading these intermediate data stored in the LRI memories at each time. M of the low resolution images synthesized by the synthesiser 60 may also be temporarily stored in the LRI memory.

The inter-transmission synthesiser 61 performs inter-transmission aperture synthesis by reading out the weight for each transmission stored in the inter-transmission aperture synthesis memory 64, weighting the synthesized images for M times of transmission stored in a memory in the synthesiser 60 or the LRI memory 56, and adding them.

In the inter-transmission synthesiser 61, the synthesized low resolution image obtained for every one time of transmission may be weighted for every transmission, and added, and only the intermediate added image obtained after the addition may be stored in the memory. By repeating M times the operation of weighting synthesized low resolution image of the following transmission and adding them for the above intermediate image to perform calculation only for the portions updated in every transmission, M of synthesized images are synthesized. If such processings as described above are performed, the memory may store only one intermediate image at most, which memory otherwise must store M of synthesized images beforehand, and therefore information amount to be memorized for that point can be reduced to 1/M.

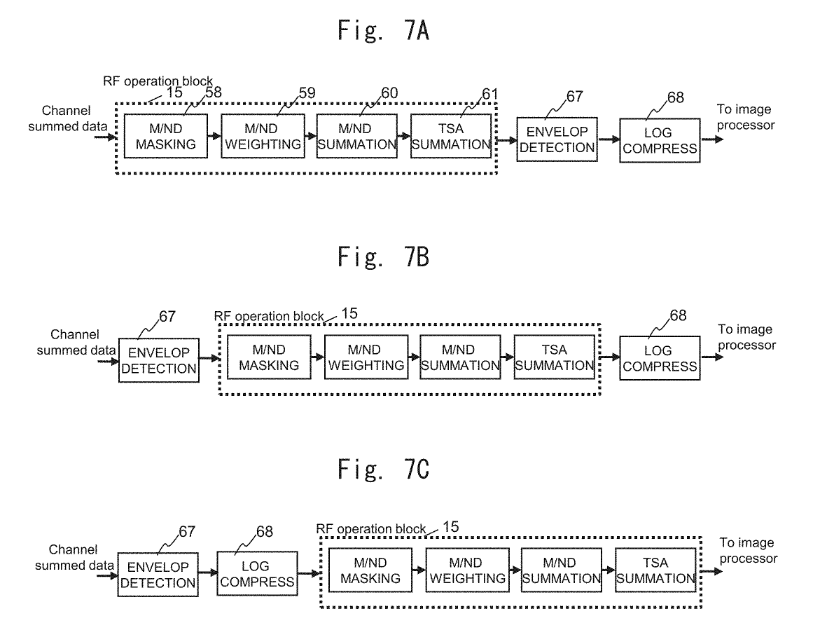

In addition to the configuration shown in FIG. 5, the RF signal processor 15 has an envelope demodulator 67 and an LOG compressor 68 as shown in FIG. 7A. Since the aperture-synthesized image (phased signals) still contains frequency components at the time of the transmission, envelope demodulation is performed with the envelope demodulator 67, and LOG compression is performed with the LOG compressor 68. The obtained image (phased signals) is sent to the image processor 109.

The image processor 109 performs predetermined image processings under control by the controller 111, and makes the image display 103 display the image.

The phasing parameter calculator 16 comprises a multi-line reception focus calculator 17, a synthesis area calculator 18, and a synthesis weight calculator 19.

The controller 111 comprises a transmission beam shape calculator 20 that receives transmission conditions such as position of the transmission focus 30, transmission frequency, imaging area, and number of times of transmission from the controller 111, and obtains geometrical shape and position of the transmission beam (direct beam) 31 by calculation. The multi-line reception focus calculator 17 receives a transmission beam shape from the transmission beam shape calculator 20, sets a plurality of reception scanning lines in the imaging area for every transmission (refer to FIG. 4), and sets a plurality of reception focuses on the reception scanning lines with predetermined intervals. Then, three kinds of factors, delay time for the direct beam 31 for phasing the received signals for each reception focus on the set reception scanning lines, delay time for the non-direct beam 33-1, and delay time for the non-direct beam 33-2 are obtained by calculation. Specifically, the delay time for the direct beam 31 is calculated by an approximation calculation method based on a geometrical sound wave propagation model such as the known virtual sound source method for obtained delay time using the transmission focus 30 as a virtual sound source. The delay time for the non-direct beam 33-1 is calculated by a known delay time calculation method for a spherical wave that propagates from the transmission channel 105-1 at one end of the ultrasonic device array 101 as a sound source. The delay time for the non-direct beam 33-2 is calculated by a known delay time calculation method for a spherical wave that propagates from the transmission channel 105-2 at the other end of the ultrasonic device array 101 as a sound source. The calculated delay times are stored in the focus memory 55.

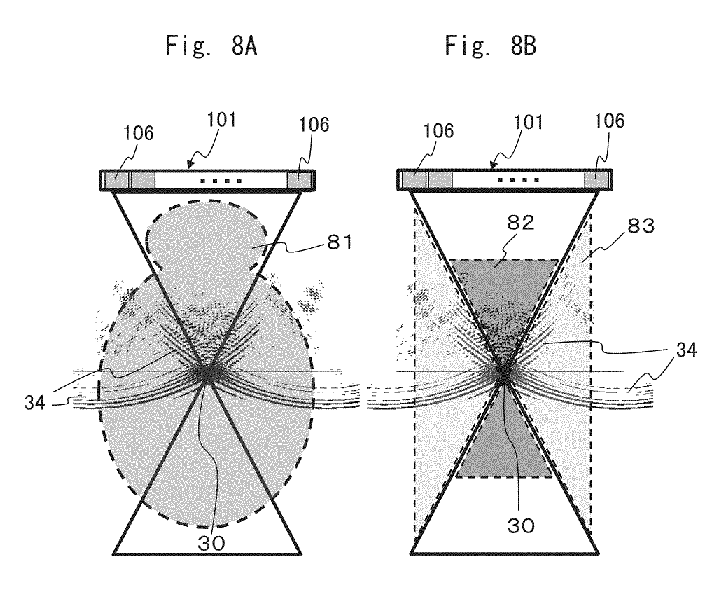

The synthesis area calculator 18 generates the mask 67 for direct beam, and the masks 68-1 and 68-2 for non-direct beam on the basis of the geometrical shape of the transmission beam 31 calculated by the transmission beam shape calculator 20, and the region for which the synthesis should be performed. The region for which the synthesis should be performed is a region for which the phased signals derived from the direct beam 31, and non-direct beams 33-1 and 33-2 should be synthesized, and for example, either one of such regions 81, 82, and 83 as shown in FIGS. 8A and 8B determined beforehand as a region for which the synthesis should be performed can be chosen and used. It is also possible to receive a region selected from the regions 81, 82, and 83 for which the synthesis should be performed from an operator, or an arbitrary shape may be received from an operator and used as a region for which the synthesis should be performed.

For example, the synthesis area calculator 18 sets the shapes of the mask 67 for direct beam and masks 68-1 and 68-2 for non-direct beam so that the phased signals based on the direct beam 31 and the non-direct beams 33-1 and 33-2 are synthesized in the region 81 around the transmission focus 30 shown in FIG. 8A, only the phased signals based on the direct beam 31 are used in a region locating outside the region 81 and inside the geometrical shape of the transmission beam 31, and phased signals based on the non-direct beams 33-1 and 33-2 are used, or any phased signals are not used for a region locating outside the region 81 and outside the geometrical shape of the transmission beam 31. The set masks 67, 68-1, and 68-2 are stored in the mask memory 62.

Further, for example, as shown in FIG. 8B, the shapes of the mask 67 for direct beam and masks 68-1 and 68-2 for non-direct beam can be set so that, in a region 82 locating inside the shape of the transmission beam 31 and near the transmission focus 30, the phased signals based on the direct beam 31 and the non-direct beams 33-1 and 33-2 are synthesized, and in a region other than the foregoing region, the phased signals based on the direct beam 31 or the non-direct beams 33-1 and 33-2 are used as in the case of FIG. 8A. Furthermore, the shapes of the mask 67 for direct beam and masks 68-1 and 68-2 for non-direct beam may be set so that, in a region locating outside the shape of the transmission beam 31 and near the transmission focus 30, such as the region 83 shown in FIG. 8B, the phased signals based on the direct beam 31 and the non-direct beams 33-1 and 33-2 are synthesized.

The weight calculator 19 sets weight values, weight distribution 70 for direct beam and weight distributions 71-1 and 71-2 for non-direct beam that show relation with a region to which the weight values are applied by using a predetermined weight calculation method such as a function for weighting according to the geometrical shape of the transmission beam 31, and the distance between the transmission focus 30 and the reception focus. The obtained weight distributions 70, 71-1, and 71-2 are stored in the weight memory 63.

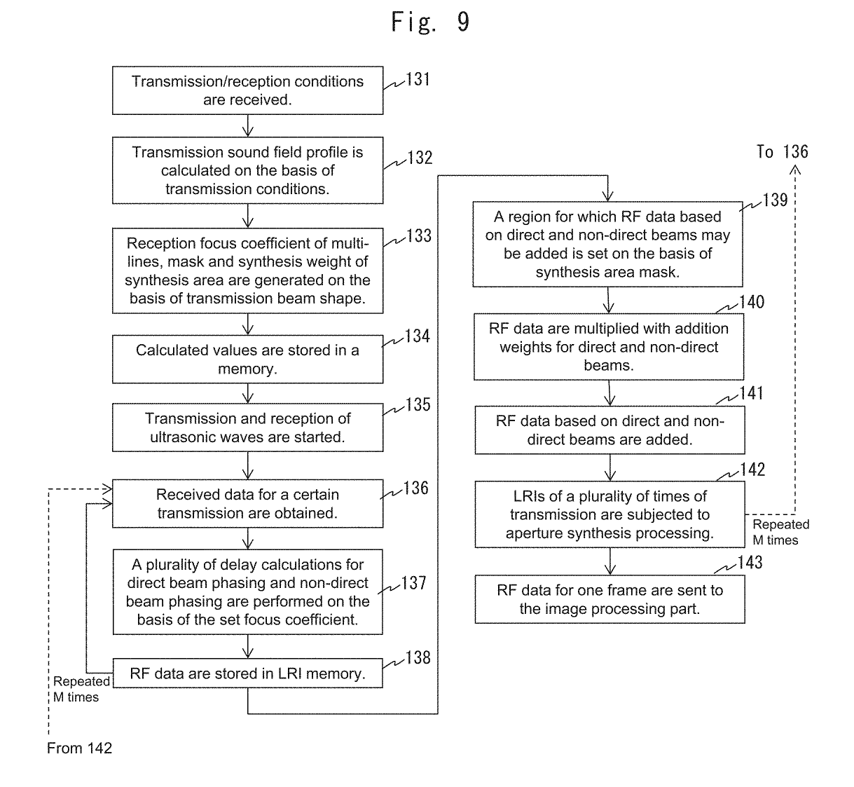

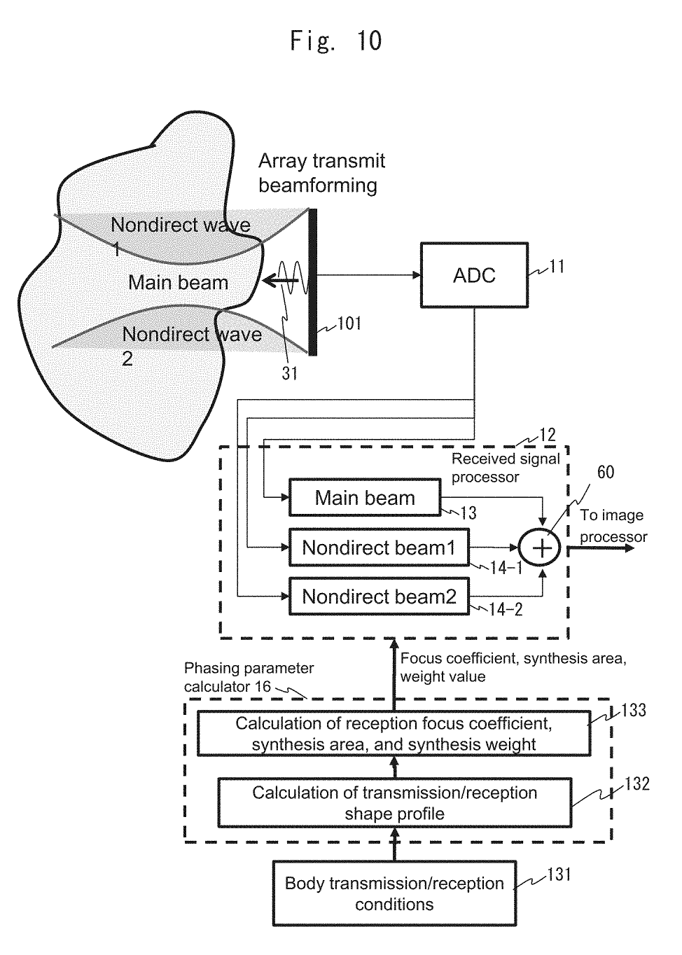

Hereafter, operations of the ultrasonic imaging apparatus of this embodiment at the time of imaging will be explained with reference to FIGS. 9 and 10.

First, the controller 111 receives transmission and reception conditions such as position of the transmission focus 30, transmission frequency, imaging area, and number of times of transmission via the console 110 (Step 131).

The transmission beam shape calculator 20 of the controller 111 calculates the shape of transmission beam 31 on the basis of the conditions received in the step 91 (step 132). The multi-line reception focus calculator 17, synthesis area calculator 18, and synthesis weight calculator 19 set a predetermined number (N) of reception scanning lines 36 by using the shape of transmission beam 31 calculated in the step 92, and so forth (refer to FIG. 4), set a plurality of reception focuses on each reception scanning line 36, calculate three kinds of delay times for each reception focus, three kinds of the masks 67, 68-1, and 68-2, and synthetic weight distributions 70, 71-1, and 71-2, and stores them in the focus memory 55, mask memory 62, and weight memory 63, respectively (steps 133 and 134)

The controller 111 sends the transmission conditions such as position of the transmission focus 30, transmission frequency, and number of times of transmission to the transmission beamformer 104, and makes the ultrasonic device array 101 transmit ultrasonic waves from the transmission channels 105 (step 135).

The reception channel 106 of the ultrasonic device array 101 receives sound waves from the subject produced by the transmission in the step 135, and outputs reception signals (step 136).

N of the delay circuit sets 51 of the first delayer 13 of the reception signal processor 12 delay reception signals for each reception channel 106 with a delay circuit of K channels built in each of them, and adds the reception signals of each channel with the adder 53 to give phased signals (RF data) based on the direct beam 31.

In this operation, as the delay time, the delay time for direct beam 31 stored in the focus memory 55 for each reception scanning line is used. Delay and addition are similarly performed also in the second delayers 14-1 and 14-2 to obtain phased signals (RF data) based on the non-direct beams 33-1 and 33-2 (step 137). The obtained phased signals based on the direct beam 31, and the phased signals based on the non-direct beams 33-1 and 33-2 are stored in the LRI memory 56 for each reception scanning line (step 56). As a result, the low resolution image 65 based on the direct beam 31, and the low resolution images 66-1 and 66-2 based on the non-direct beams 33-1 and 33-2 are stored. The steps 136 to 138 are repeated for each of M times of transmission.

The synthesis area setter 58 sets the masks 67, 68-1, and 68-2 for the phased signals (low resolution images 65, 66-1, and 66-2), and sets a region for which the phased signals based on the direct beam 31, and the phased signals based on the non-direct beams 33-1 and 33-2 may be added (step 139). The masks 67, 68-1, and 68-2 are read out from the mask memory 62, and used.

The weighting part 59 carries out weighting of the masked phased signals 31, 33-1, and 33-2 (step 140). As the weight values, the values of the weight distributions 70, 71-1, and 71-2 stored in the weight memory 63 are used.

There may also be employed a configuration that, in the inter-transmission synthesiser 61, the low resolution images synthesized in each transmission are weighted and added after every transmission, and only the added intermediate addition images are stored in a memory. By repeating M times the operation of weighting the synthesized low resolution images of the subsequent transmission and adding them for the above intermediate images to perform calculation only for the portions updated in every transmission, M of synthesized images are synthesized. In this case, the steps 136 to 142 are successively performed for reception data of a certain transmission, and the steps 136 to 142 are repeats M times (loop indicated with dashed line in FIG. 9). If such processings as described above are performed, the memory may only store one intermediate image at most, which memory otherwise must store M of synthesized images beforehand, and therefore information amount to be memorized for that point can be reduced to 1/M.

The synthesiser 61 adds the weighted phased signals based on the direct beam 31 (low resolution image 65), and the weighted phased signals based on the non-direct beams 33-1 and 33-2 (low resolution images 66-1 and 66-2) (Step 141) to synthesize them. The steps 139 to 141 are repeated for all the phased signals (low resolution images) of M times of transmission.

The inter-transmission aperture synthesiser 61 performs weighting of the synthesized phased signals (low resolution images) for each of M times of transmission with the weights stored in the weight memory 64, and then adds them to obtain inter-transmission aperture-synthesized phased signals (images) (step 142). The obtained phased signals are subjected to envelope demodulation and LOG compression, and then sent to the image processor 143 (step 143). The image processor 143 performs desired image processings of the signals, and makes the image display 103 display the obtained image.

Since the information based on the direct beam 31 and the information based on the non-direct beams 33-1 and 33-2 are synthesized in the displayed image with the synthesiser 60, and the inter-transmission aperture synthesis is also performed, the displayed image has high resolution. Further, since a plurality of reception scanning lines can be set for one time of transmission, a high resolution image can be obtained with a small number of times of transmission, and thus high-speed imaging is enabled.

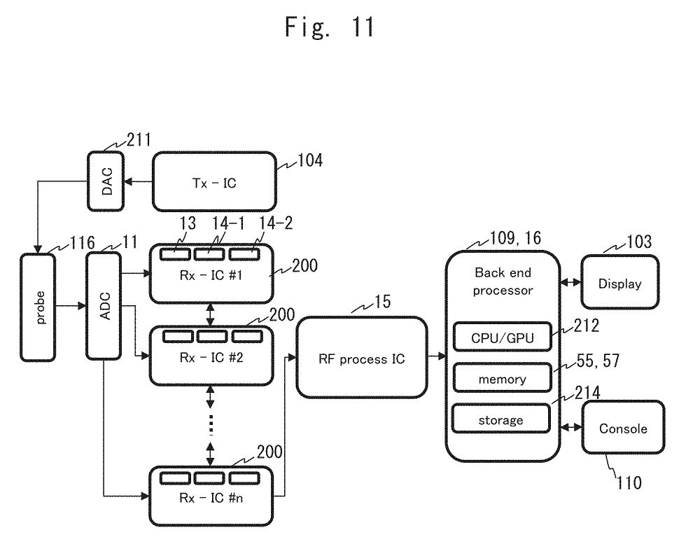

The configuration of the ultrasonic diagnostic apparatus of the second embodiment described above used when it is realized with hardware is shown in FIG. 11.

There is employed a configuration that the transmission beamformer is constituted with integrated circuits (Tx-ICs), and connected to the ultrasound probe 116 via a digital/analogue converter 211. The delayers 13, 14-1, and 14-2 are constituted with one or more integrated circuits 200 (Rx-ICs). The integrated circuits 200 (Rx-ICs) comprise the delay circuit set 51 of a predetermined number of channels, and the adder 53 for adding the output of the delay circuit set 51. The delayers 13, 14-1, and 14-2 of K channels can be constituted by parallel arrangement of the integrated circuits 200 (Rx-ICs) in a number of J, which is a number smaller than K. The delayers 13-1, 14-1 and 14-2 can be constituted with a logic circuit (Rx-IC) contained in each of the integrated circuits 200. Outputs of the integrated circuits 200 (Rx-ICs) are cascade- or daisy chain-connected, and sent to an integrated circuit 15 (RF process IC) of the subsequent stage as N of reception beams of K channels.

The integrated circuit 15 (RF process IC) contains a circuit that operates as the synthesis area setter 58, the weighting part 59, the synthesiser 60, and the inter-transmission aperture synthesiser 61. For these integrated circuits (Rx-ICs and RF process IC), ASIC (application specific integrated circuit), FPGA (field-programmable gate array), and so forth can be used.

The functions of the image processor 109, phasing parameter calculator 16, and memories 55 and 57 can be realized with CPU 212, a memory 213, and a storage 214. That is, there is employed a configuration that CPU 212 reads and executes programs stored in the storage 214 beforehand to realize the operations of the steps 132 to 134 shown in FIG. 9.

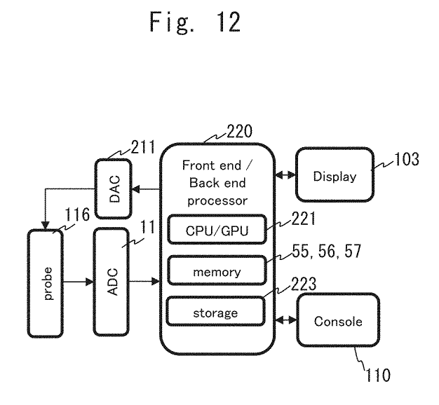

The configuration of the ultrasonic diagnostic apparatus of the second embodiment used when it is realized with software is shown in FIG. 12. As shown in FIG. 12, the ultrasonic diagnostic apparatus comprises the probe 116, CPU (or GPU, or both of CPU and GPU) 221, memories 55, 56, and 57, and a storage 223, and CPU 221 reads and executes programs stored in the storage 223 to perform the steps shown in FIG. 9. The transmission beamformer 104, the reception signal processor 12, the controller 111, and the phasing parameter calculator 16 can be thereby realized with software.

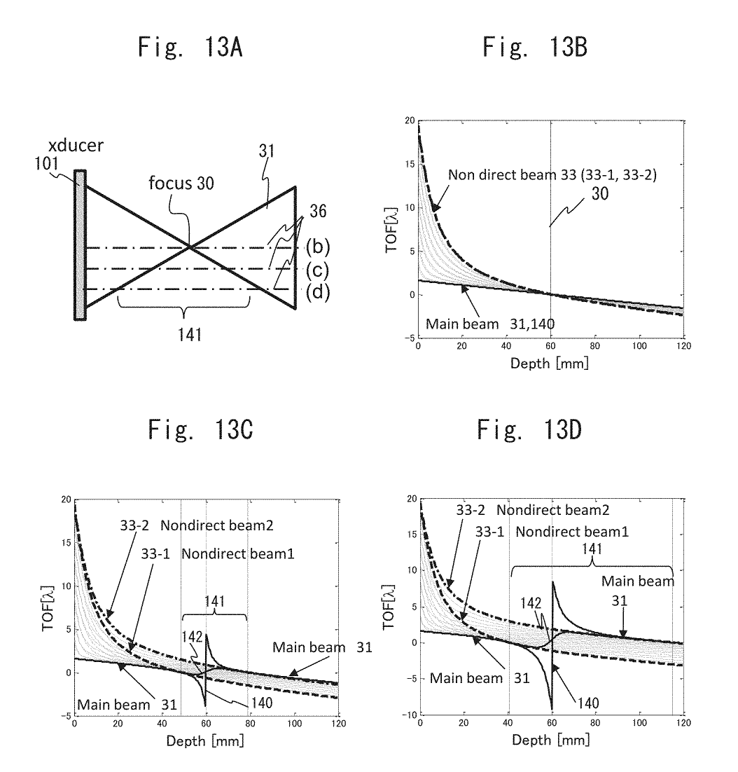

The effect of this embodiment that the information based on the direct beam 31, and the information based on the non-direct beams 33-1 and 33-2 can be synthesized with the synthesiser 60 will be explained in detail with reference to FIGS. 13A to 13D. FIG. 13A shows the shape of the transmission beam (direct beam) 31, the transmission focus 30, and three of the reception scanning lines 36, and FIGS. 13B to 13D are graphs showing time until arrival of the direct beam and non-direct beams from about 100 of transmission channels for the depth after transmission of the transmission beam 31, in which the vertical axes indicate the time until arrival (=propagation distance), and the horizontal axes indicate the depth from the ultrasonic device array 101.

FIG. 13B shows the time until the direct beam and the non-direct beam arrive at various positions on the reception scanning line 36 coinciding to the sound axis.

As shown in FIG. 13B, at a position near the ultrasonic device array 101, the direct beam 31 arrives first, and the non-direct beam 33 arrives with significant delay.

However, it can be seen that, as the depth becomes larger, the difference of the times until arrival of the direct beam 31 and the non-direct beam 33 sharply became smaller, and they arrive at the transmission focus 30 at the same time. However, at a position other than the transmission focus 30, the times until arrival of the sound waves transmitted from each of the transmission channels 105 fluctuate, and cover a wide range. Therefore, phasing cannot be carried out with the delay time matched with the time until arrival of the direct beam 31. However, according to this embodiment, the delayers 14-1 and 14-2 perform phasing with a delay time matched with the non-direct beams 33-1 and 33-2, and therefore the non-direct beams 33-1 and 33-2 can also be phased.

FIGS. 13C and 13D show times until arrival of the direct beam and the non-direct beam at various positions on the reception scanning line 36 at a position remote from the sound axis. It can be seen that, as the reception scanning line 36 becomes remoter from the sound axis, variation of the time until arrival of the sound wave becomes larger. In the region 141 locating outside the transmission beam 31, the direct beam 31 does not reach, and the curve of the delay time 140 obtained by the virtual sound source method is disrupted around the transmission focus 30. That is, this region cannot generally be used for imaging.

As seen from FIGS. 13C and 13D, the non-direct beams 33-1 and 33-2 propagate also to the region 141. Therefore, in the region 141, reception beamforming (phasing addition) can be performed by phasing the non-direct beams 33-1 and 33-2 with delay times defined according to times until arrival of them as in the aforementioned embodiment.

In order to secure the continuity of ultrasonogram around the focus 30, in the region 141, delay time 142 obtained by approximation may be used as substitution for the delay time of the transmission beam 31 as shown in FIGS. 13C and 13D.

In the above explanation of this embodiment, the non-direct beams 33-1 and 33-2 are explained as representative sound waves transmitted from the transmission channels 105-1 and 105-2 locating at the both ends of the ultrasonic device array 101. However, since sound waves have a spatial energy density, a non-direct beam of large energy may be determined, and delay time may be determined from the time until arrival of that wave.

Further, at a position of a large energy density, there are offsets from the non-direct beams 33-1 and 33-2, for which the devices at the both ends are assumed.

Therefore, the offsets may be obtained beforehand, and spherical waves transmitted from devices shifted by the offsets (devices locating slightly inside or outside the both ends) may be used as the non-direct beams 33-1 and 33-2. These offsets may be defined as a function, and calculated in the apparatus, or may be described in the form of a table and stored beforehand in a memory contained in the apparatus.

In the above-explained configuration shown in FIG. 7A, the envelope demodulator 67 and the LOG compressor 68 are disposed as subsequent stages of the RF signal processor 15. However, this embodiment is not limited to the configuration shown in FIG. 7A. Only the envelope demodulator 67 may be disposed as a preceding stage of the RF signal processor 15 as shown in FIG. 7B, or both the envelope demodulator 67 and the LOG compressor 68 may be disposed as preceding stages of the RF signal processor 15 as shown in FIG. 7C.

### Third Embodiment

In the second embodiment explained above, the phased signals based on the direct beam 31 and the phased signals based on the non-direct beams 33-1 and 33-2 are added with the synthesiser 60 disposed as a subsequent stage of the adders 53, 54-1, and 54-2 for the phased signals of each channel of the reception beamformer 108 as shown in FIG. 5.

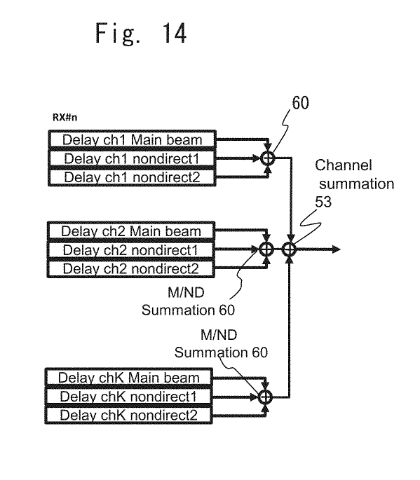

However, the present invention is not limited to this configuration. In the third embodiment, as shown in FIG. 14, the synthesisers 60 in a number of K are disposed in the reception beamformer 108, and are made to add outputs of the delay circuits of corresponding channel numbers of the delay circuit set 51 for direct beam and the delay circuit sets 52-1 and 52-2 for non-direct beam.

Synthesized delayed signals for K of the channels are thereby obtained, and therefore the synthesized and delayed signals of the K channels are added in the inter-channel adder 53 disposed as a subsequent stage.

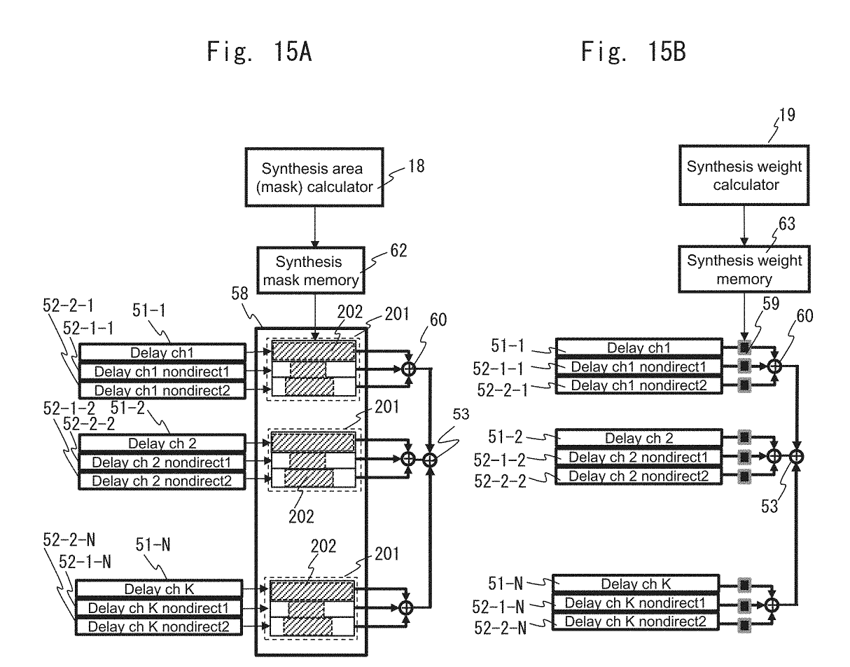

When the synthesis area setter 58 is disposed in this configuration, the synthesis area setter 58 is disposed as a subsequent stage of the delay circuit sets 51, 52-1, and 52-252, as shown in FIG. 15A. In this case, a synthetic mask 202 is provided for delayed data 201.

When the weighting part 59 is disposed, it may be disposed between the inter-channel adder 53 and the delay circuit sets 51, 52-1 and 52-2, as shown in FIG. 15B.

Since the other configurations are the same as those of the second embodiment, explanations thereof are omitted.

### Comparative Example

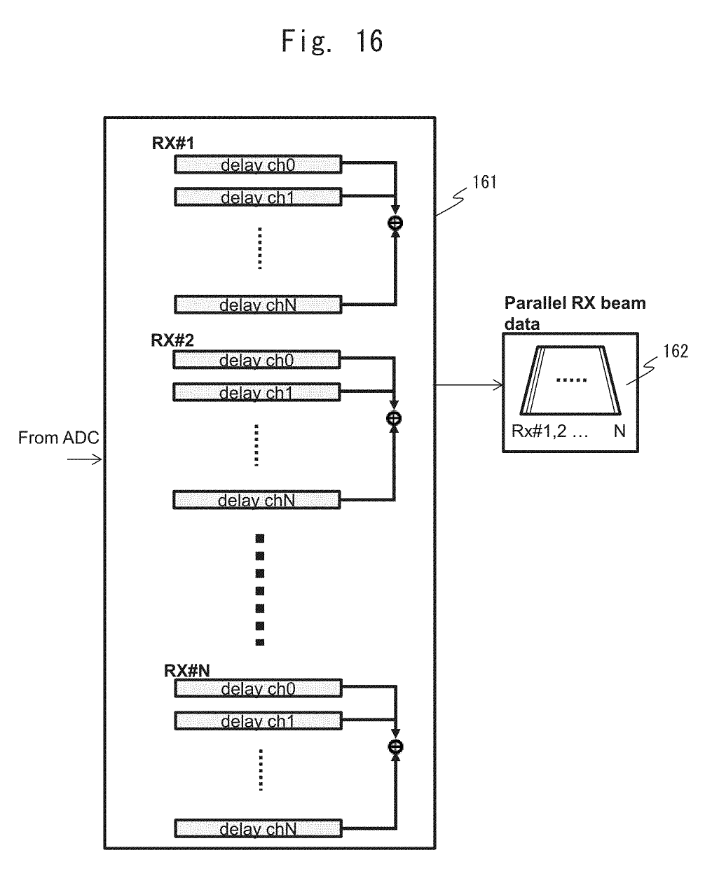

As a comparative example, a configuration that LRI (low resolution image) 162 is generated from a plurality of reception beams by the conventional parallel beam processing is shown in FIG. 16. By comparison of FIG. 16 with the second embodiment and FIG. 5, it can be seen that the delayer 161 of the comparative example shown in FIG. 16 corresponds to the delayer 13 for the direct beam 31 shown in FIG. 5. Further, only one kind of LRI 162 is generated, unlike the case shown in FIG. 5. That is, it can be seen that the configuration of this embodiment shown in FIG. 5 is completely different from that of the comparative example in that the delayers 14-1 and 14-2 for the non-direct beams 33-1 and 33-2, which are not provided in conventional apparatuses, are provided in parallel with the delayer 13, and the delay processing is simultaneously performed.

### DESCRIPTION OF NUMERICAL NOTATIONS

101 Ultrasonic device array
102 Body of ultrasonic imaging apparatus
103 Image display
104 Transmission beamformer
106 Ultrasound probe
107 Transmission/reception separation circuit (T/R)
108 Reception beamformer
109 Image processor
110 Console
111 Controller

1. An ultrasonic imaging apparatus comprising a received signal processor that processes received signals obtained by receiving sound waves from a subject, to which a transmission beam imparted with such a predetermined phase delay that it focuses on a predetermined transmission focus has been transmitted, with a plurality of reception channels to obtain phased signals, wherein:
the received signal processor comprises two or more delayers disposed for each of the plurality of the reception channels, and a synthesiser,
among two or more of the delayers, a first delayer delays the received signals by a first delay time for phasing the received signals produced from the transmission beam for a predetermined reception focus, a second delayer delays the received signals by a second delay time for phasing the received signals produced from a sound wave of a predetermined phase different from the phase of the transmission beam for the same reception focus, and
the synthesiser adds first phased signals generated by the first delayer through the delaying, and second phased signals generated by the second delayer through the delaying.

2. The ultrasonic imaging apparatus according to claim 1, wherein the received signal processor sets a plurality of reception scanning lines in the inside and outside of a transmission region of the transmission beam of the subject for one time of transmission of the transmission beam, and sets a plurality of reception focuses on the reception scanning lines.

3. The ultrasonic imaging apparatus according to claim 1, wherein the synthesiser performs weighting when it synthesizes the first phased signals and the second phased signals.

4. The ultrasonic imaging apparatus according to claim 3, wherein weights used for the weighting are set on the basis of positional relationship of the reception focuses and a transmission region of the transmission beam.

5. The ultrasonic imaging apparatus according to claim 1, wherein the received signal processor further comprises a synthesis area setter that sets a region of the subject for which the first phased signals and the second phased signals should be added, and the synthesiser adds the first phased signals and the second phased signals for the reception focuses in the region set by the synthesis area setter.

6. The ultrasonic imaging apparatus according to claim 1, which further comprises a delaying amount calculator that obtains the first delay time and the second delay time by calculation using positional relationship of the shape and the transmission focus of the transmission beam, and the reception focuses.

7. The ultrasonic imaging apparatus according to claim 1, wherein the received signal processor has a first adder that adds the first phased signals delayed by the first delayer for the every reception channel, and a second adder that adds the second phased signals delayed by the second delayer for every reception channel, and the synthesiser synthesizes the first phased signals added by the first adder and the second phased signals added by the second adder.

8. The ultrasonic imaging apparatus according to claim 1, wherein the synthesiser adds the first phased signals outputted by the first delayer and the second delayer, both of which are disposed at a predetermined reception channel, and
the received signal processor further comprises an adder that further adds the phased signals added by the synthesiser between reception channels.

9. The ultrasonic imaging apparatus according to claim 7, wherein the received signal processor comprises an envelope demodulator that performs envelope demodulation of signals as one of preceding stage and subsequent stage of the synthesiser.

10. The ultrasonic imaging apparatus according to claim 8, wherein the received signal processor comprises an envelope demodulator that performs envelope demodulation of the phased signals as one of preceding stage and subsequent stage of the synthesiser.

11. A method for processing ultrasonic wave signals comprising:
transmitting a transmission beam imparted with a predetermined phase delay so that it focuses on a predetermined transmission focus to a subject,
receiving sound waves from the subject with a plurality of reception channels,
delaying received signals received by the reception channels by a first delay time for phasing the received signals produced from the transmission beam for a predetermined reception focus, delaying the received signals by a second delay time for phasing the received signals produced from a sound wave of a predetermined phase different from the phase of the transmission beam for the same reception focus, and
adding first phased signals obtained by the delaying by the first delay time, and second phased signals obtained by the delaying by the second delay time.
